(19) United States
(12) Patent Application Publication
(10) Pub. No.: US 2013/0273609 A1
(43) Pub. Date: Oct. 17, 2013

(54) PRIMER BEADS

(71) Applicants: Nam Quoc Ngo, Los Gatos, CA (US); Hoc Thai Nguyen, Bac Lieu Province (VN); Minh Tri Thi Dang, San Jose, CA (US); Ngoc Dieu Ngo, San Jose, CA (US); Laurent Jaquinod, Davis, CA (US)

(72) Inventors: Nam Quoc Ngo, Los Gatos, CA (US); Hoc Thai Nguyen, Bac Lieu Province (VN); Minh Tri Thi Dang, San Jose, CA (US); Ngoc Dieu Ngo, San Jose, CA (US); Laurent Jaquinod, Davis, CA (US)

(73) Assignee: Chemistry and Technology For Genes, Inc., San Jose, CA (US)

(21) Appl. No.: 13/805,498

(22) PCT Filed: Jun. 30, 2011
(86) PCT No.: PCT/US11/42696

§ 371 Date: Jun. 10, 2013

Related U.S. Application Data
(60) Provisional application No. 61/398,666, filed on Jun. 30, 2010.
(60) Provisional application No. 61/455,855, filed on Oct. 28, 2010.

(51) Int. Cl. C12P 19/34

(52) U.S. Cl. CPC C12P 19/34
USPC 435/91.2; 435/262; 435/194

(57) ABSTRACT

Non-hydrophobic beads and methods to reversibly bind, normalize, store and in situ deliver primers to reactions including PCR. Also provided are instructions for preparing the beads. In the presence of an appropriate binding buffer, a bead can be used to bind and desalt primers from a crude solution of DMT-off primers. In the presence of an appropriate binding buffer, a bead can be used to bind and purify primers from a crude solution of DMT-on primers. A bead may bind a picomolar amount of DMT-on primers from a solution containing a plurality of crude DMT-on primers. Upon detritylation and washing, the resulting DMT-off primer bound bead may be used in PCR. Primers are released from the bead upon cycling the temperature. Primer bound beads are coated or silanized with hydrophobic reagents which ensures a gradual release of primers during the thermal cycling of the PCR reaction. Coating or silanization in turn enhances primer stability and long term storage.

### TECHNICAL FIELD

The present invention relates to the fields of nucleic acid chemistry and molecular biology, and more specifically to reagents and methods facilitating the purification, normalization and storage of primers and their delivery and release to amplification reactions.

### BACKGROUND

Currently, enzymatic amplification reactions, such as the polymerase chain reaction (PCR), are widely used. This ubiquity provides incentives to streamline enzymatic reaction assembly in order to improve yield and quality of the reaction products and to reduce costs.

Reagents used in PCR have been freeze-dried (U.S. Pat. No. 5,834,254) or encapsulated thus making an amplification reaction set up as simple as adding water, target DNA template and primers. For instance, a “Ready-To-Go RT-PCR Bead” from GE Healthcare contains all the components needed for an amplification reaction besides template RNA and primers.

Various formats of primers delivery have been proposed to minimize liquid handling and cross-contaminations. Biotin-labeled primers immobilized on streptavidin-coated surfaces (Westin et al.) or primers covalently immobilized to solid supports (U.S. Pat. No. 7,582,470) were described to amplify complementary target sequences. U.S. Pat. No. 7,615,193 describes a robotic delivery of beads into PCR wells but does not disclose the type of beads, the bead surface compositions and the methods used in preparing primer bound beads.

Amplification reaction mixtures are typically assembled at temperature below the temperature needed to yield primer hybridization specificity and result in primer extension in competition with the amplification of the target sequences (Chou et al., 1992). This significantly decreases the efficiency of the amplification of the target sequence, notably when samples containing low copies of templates are amplified, and is a major limitation of PCR. In “hot-start” amplifications, one or more reagents are withheld from the reaction mixture or chemically or enzymatically blocked until a temperature providing the necessary hybridization specificity is reached. This suppresses primer extension during the time of low primer hybridization specificity and minimizes primer-dimer formation. To limit amplification to temperatures wherein the primers are known to hybridize mostly to the intended target sequence, a reagent can be withheld and added to the reaction wells after the initial high temperature incubation step. Although effective, this first hot-start approach was cumbersome and soon replaced by a physical separation of reaction components with a heat sensitive material such as wax. Upon melting of the heat sensitive material during the denaturation step, all reagents needed to start an amplification reaction are allowed to mix (U.S. Pat. No. 5,411,876). Other hot-start methods rely on the sequestration of magnesium (WO 2003012066), heat activation of an enzyme (U.S. Pat. No.5,773,258 and U.S. Pat. No. 5,677,152) or on reversible inhibition of the polymerase by polymerase-specific antibodies. Amplification reaction begins when the antibodies are inactivated by elevated temperatures (U.S. Pat. No. 5,338,671). To block primer extension, primers have been blocked by forming hairpin loop (U.S. Pat. No. 5,866,336) or by protecting the 3′-hydroxy group with a heat-labile protecting group (U.S. Pat. No. 6,509,157; Lebedev et al., 2008). Non-specific amplifications are reduced because the primers do not support primer extension until temperature insuring primer hybridization specificity is reached. Drawbacks of current hot-start methods stem from their reliance on expensive primers, specific polymerase or antibodies; or on limited throughput.

There is a need to achieve a non manual “hot-start” PCR which is reliable, simple and economical.

### SUMMARY

The above and other objects are achieved in a method in which a bead is used for introducing an oligonucleotides into a reaction. The bead has normalized, non-covalently bound oligonucleotides, on a bead surface. The bead then gradually releases the oligoneucleotides from the bead during an enzymatic reaction, such as PCR. An additional initial step could include preparation of the bead by purifying (e.g. desalting) a crude solution of oligonucleotides by capture of a normalized amount of purified oligonucleotides on the bead. In this method the oligonucleotides could be picomolar amounts of a primer.

The bead, in some embodiments is a porous or nonporous glass bead having a nonpolar, hydrophobic surface. These beads may have non-polar hydrophobic surface including linear or branched alkyl chains selected from C₃ to C₂₀, aryl, benzyl, naphtyl, phenanthryl and trityl groups. The initial step of preparing the beads may include reacting glass beads with silanes. In some embodiments, the beads include a non-polar hydrophobic surface including surface (Aryl)ₙ-X-alkylgroups wherein said Aryl is selected from a group consisting of phenyl, benzyl, biphenyl, naphtyl, trityl, X=C, O, S, SC(O)N, OC(O)N, NC(O), and N; n=1 to 3, n being a finite integer equal or greater than one. In some embodiments the oligionucleotides are purified by binding oligonucleotides having a 5′-hydrophobic moiety from the crude solution containing at least one contaminant.

This 5′-hydrophobic moiety may be a 4,4′-dimethoxytrityl group (DMT) and said contaminant may be a DMT-off nucleic acid. The oligonucloetides on the beads may include primers (e.g. at least one reverse primer and a forward primer) and/or a nucleic acid probe. The method could also include using a binding buffer and mixing a resulting solution with a plurality of primed beads yielding DMT-on oligonucleotide bound bead. This could be followed by washing the DMT-on oligonucleotide bound bead with a washing buffer that removes contaminants but not bound DMT-on oligonucleotides. An example of capture of a normalized amount of purified oligonucleotides on said bead may include exposing said bead to a solution containing 10 to 100% of methanol, ethanol, acetonitrile or acetone in water; and subsequently exposing said bead to a solution containing 0.1M to 1.0 M concentration of monoalkylammonium, dialkylammonium, trialkylammonium acetate at pH ranging from 6 to 9.5. The method of claim 15, further comprising a step of cleaving DMT groups of the said bound DMT-on oligonucleotide yielding DMT-off oligonucleotide bound bead.

### BRIEF DESCRIPTION OF THE DRAWINGS

FIG. 1 is a modified chemical diagram showing the functional groups covalently bound to the bead surfaCe.

FIG. 2 is a modified chemical diagram showing the preparation of a (tritylmercaptopropyl) bead or (T-bead).

FIG. 3 is a chromatogram chart from an Ion exchange HPLC of an amplification reaction of 100 copies of a 106-mer template using primers that have been pipeted in a PCR buffer.

FIGS. 4a and 4b are chromatogram charts from are Ion exchange HPLC of a amplification reactions of (a) 10 copies and (b) 100 copies of a 106-mer template using primers released in situ by a silanized primer bound bead.

### DETAILED DESCRIPTION

Generic terms used herein are defined as follows: “nucleic acid” and “oligonucleotide” refer to polydeoxyribonucleotides or polyribonucleotides and include single-stranded DNA and single-stranded RNA.

“primer” refers to an oligonucleotide that is complementary to a DNA template to be amplified in an appropriate amplification buffer.

“amplification buffers” are solutions containing notably a DNA polymerase, typically a thermostable DNA polymerase, and four different nucleoside triphosphates to which are added primers at the outset of an amplification reaction. Preferably, primers used in amplification processes are single-stranded and ranging from 10 to 50 nucleotides. The term primer refers also to oligonucleotide containing modified purine or pyrimidine base, nucleotide analogs, or additional nucleic acid sequence covalently attached at the 5′-end. In addition, a primer can be modified with a detection label such as fluorescein, rhodamine, and cyanine, a linker such as an alkylamine or a reporter such as biotin.

“primer-dimer” refers to template independent artifacts of amplification reactions such as those resulting from primer extensions wherein another primer serves as a template.

Low cost low throughput PCR machines are being developed to be used by unskilled physicians (Neuzil et al.) but fewer efforts are aimed on making the PCR assembly cheaper and reliable. The bead described herein fulfills this need by facilitating the end-user handling of its primers. The steps of diluting, normalizing and pipeting primer stock solutions are eliminated prior assembly of PCR reactions thus reducing the risk of contamination and the waste of unused solutions of primers. Primer normalization takes place during the purification or desalting process, wherein each bead binds the targeted picomolar amount of primers needed to run an amplification reaction. For example, a bead is used to bind, normalize and purify DMT-on primers. The binding of DMT-on primers to a bead takes advantage of known trityl-on purification techniques (Cashion et al, 1973). DMT-on primers can be synthesized by any method for synthesizing oligonucleotides known to those skilled in the art, including both solid phase and solution phase methods provided the oligonucleotides retain a final hydrophobic moiety such as the dimethoxytrityl group. This hydrophobic moiety provides a mean to reversibly bind the DMT-on primers to the bead non-polar surface while truncated sequences and synthetic contaminants are eliminated in the flow-through and subsequent washing steps. Upon DMT removal, the resulting DMT-off primers remain bound to the beads and stable while protected from nucleases. This facilitates shipment, long term storage and field-use.

A primer-bound bead can be added directly to an amplification reaction such as PCR. One embodiment relates to kits for the amplification reactions of a nucleic acid target and comprises a primer bound bead, an amplification reagent (e.g., a nucleic acid polymerase or ligase), nucleoside triphosphates and suitable buffers. Beads having bound primers provide reproducibility between amplification reactions and reduce the potential for pipeting errors and contamination. The beads having bound primers offers an economical hot-start solution to the problem of non-specific amplification. Low leaching of primers from the hydrophobic bead at ambient temperature facilitates the assembly of amplification reactions. Primers are released during the preheating and denaturation steps of an amplification reaction. Eliminating room temperature leaching of primer is further achieved by coating or silanizing the primer bound beads. This in turn achieves a gradual release of primers which further minimizes primer extension by the DNA polymerase. Gradual release of primers mimics the amplification of very dilute DNA samples wherein additional stock of primers are added after few cycles in order to reduce false amplification by minimizing the early formation of non-specific primer extension products.

In one embodiment of the invention, the gradual release of primers from the beads is controlled by coating the primers bound beads with solutions containing hydrophobic reagents. In another embodiment Of the invention, the gradual release of primers from the beads is controlled by silanizing primers bound beads with solutions containing silane reagents. The said coating or silanizing steps further increase primer stability and nuclease resistance. The beads are porous, porous with a non porous core or non porous. The beads, for example, are made of porous glass beads that are silanized with alkoxysilanes bearing hydrophobic moieties and end-capped. In one embodiment the hydrophobic moieties contain aromatic groups such as trityl groups. Purification and normalization of primers take place using hydrophobized glass beads having average diameter in the 0.1 to 1.5 mm range, and preferentially around 0.5 to 1.0 mm.

The disclosed embodiments provide reagents and methods to further streamline the assembly of amplification reactions. The beads reversibly bind, normalize, store and deliver primers used in performing enzymatic amplification reactions. Nonpolar hydrophobic glass beads may be used to carry out the task of purifying crude nucleic acids while binding normalized picomolar amount of primers per bead. A primer bound bead is able to release the primers in the amplification buffer without inhibiting DNA polymerases. Further, coating or silanizing primer bound beads greatly improves the gradual release of the bound primers, offering a novel entry to hot start PCR. In the disclosed embodiments the beads, besides fulfilling the tasks of purifying, normalizing and storing primers, also provide an entry to hot start amplifications via the gradual and controlled release of primers. Primer binding takes advantage of the synthesis of nucleic acids using 5′-dimethoxytrityl protected phosphoramidite reagents and relies on the known hydrophobic binding of DMT-on nucleic acids to reversed-phase sorbents. Primer bound beads provide a means to store and protect primers. Biologist end-users can elute the nucleic acids that are reversibly bound to a bead or take advantage of the bead itself and use it in an amplification reaction.

In one embodiment, primer bound beads are used in amplification reactions and notably the polymerase chain reaction (PCR) for an in situ delivery of primers. The low leaching of primers from a primer bound bead in the amplification buffer at room temperature during the pre-amplification reaction set-up stage prevents their extension. Release of primers from a primer bound bead takes place preferentially after the reaction temperature has been raised, which insures reaction specificity and minimize primer-dimer amplification. Thus, the use of primer bound beads of the invention provides a novel entry to hot-start amplifications. In one embodiment of the invention, coating of said primer bound beads further reduces or eliminates mispriming. In another embodiment of the invention, silanization of said primer bound beads further reduce primer-dimers by preventing primer extension to take place at room temperature and by allowing the gradual release of primers in solution during the high-temperatures of the preheating step and the subsequent cycling steps of an amplification reaction.

Useful commercial kits of the invention contain any components of the amplification reaction which includes primer bound beads. Other useful commercial kits contain beads and the buffer and reagents required in the binding and desalting steps or the binding and purification steps to provide primer bound beads and the reagent needed in the coating or silanization of the resulting primer bound bead. The following aspects of the invention, bead preparation, bead binding, coating or silanization of primers bound beads and hot start amplifications of target templates are further described in detail:

### Bead Preparation:

Beads used for primer binding and delivery are made of an organic polymer (polyacrylamide, polystyrene) or preferentially an inorganic polymer (silica, glass) that is insoluble in the reagents used for PCR. Beads are porous or porous with a non porous core or non porous. Non porous beads have diameters ranging from 0.5 mm to 1.5 mm. Preferred diameter is in the 1 mm range, a diameter that facilitates a bead manual dispensing while providing sufficient primer binding capacity. Preferably, beads are made of porous glass. Porous glass beads can be manufactured in a variety of sizes. As used herein, the bead diameters are in the range of 0.05 mm to 1.5 mm and more preferably 0.1 to 1 mm. Porous means that beads contain pores having substantially similar diameter in the range between 100 to 4000 angstroms. Preferably, the pore diameters are about 500 angstroms.

Glass beads, porous or non porous, are hydrophobized by silanization with alkoxysilanes bearing hydrophobic moieties or derivatized with silanes introducing functional groups (such as CO₂H, NH₂, OH, SH) which are further reacted with hydrophobic moieties. In one embodiment, glass beads are reverse-phase materials made of controlled porous glass (CPG) covalently modified by silanes, wherein said silanes introduced non polar groups onto the bead surface. The silanes may be selected from a group including alkyltrialkoxysilanes, dialkyldialkoxysilanes, trialkylalxoxysilanes or alkylbis(trialkoxysilane). Alkyl groups include straight chained or branched hydrocarbons which are completely saturated. Alkyl groups can be substituted or unsubstituted. Suitable substituents for alkyl groups include substituted or unsubstituted aromatic groups, halogenated lower alkyl such as trifluoromethyl, —O-(alkyl or aryl groups), —S-(alkyl or aryl group), N-(alkyl or aryl groups), —NC(O)-(alkyl or aryl groups).

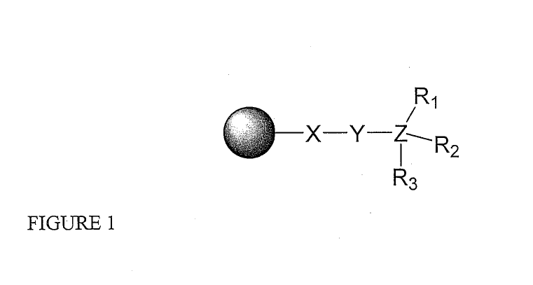

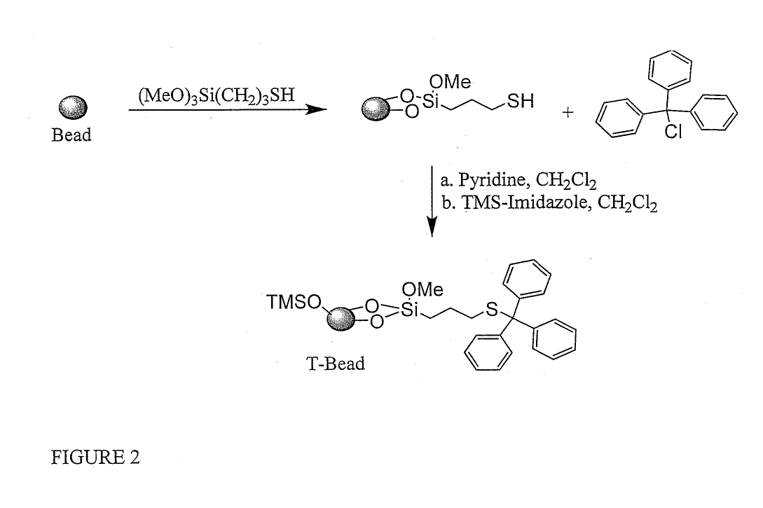

In one example, the beads of the invention are functionalized with mono-, di- and tri-aryl groups as shown in FIG. 1 wherein X=alkyl; Y=O, S, N and Z=alkyl, aryl and R₁, R₂, and R₃ represent independently an hydrogen, an alkyl, arylalkyl, cycloalkyl or an aryl group such a phenyl, napthyl, quinolyl, or other nitrogen, sulfur, and/or oxygen-containing heterocyclic ring; or an aryl groups with substituent such as halide, nitro, alkoxy, lower alkyl, and aryl. In an example, the glass beads are made of CPG modified by silanes such as mercaptoalkylsilane or aminoalkylsilane that are further derivatized with non polar, hydrophobic moieties. In an example, (aryl)ₙmethylmercaptoalkyl functionalized porous glass beads are prepared. The porous glass beads are reacted with (mercaptoalkyl)trialkoxysilane, wherein alkoxy is a methoxy or an ethoxy group or the like. The resulting (mercaptoalkyl)beads are reacted with halogenomethyl(aryl)ₙ to afford [(aryl)ₙmethylmercaptoalkyl]head, wherein (i) n=1 to 3, n being a finite integer equal or greater than one and ii) halogeno is a chloro, bromo or iodo group. The said [(aryl)ₙmethylmercaptoalkyl]heads'are further end-capped with chlorotrialkylsilane, dialkyldialkoxysilane or trialkylsilylimidazole. Alternatively, (aryl)ₙmethylmercaptoalkyl-(trialkoxy)silane is used. The trityl loading is in the range of 1 to 150 □mol/g and preferably in the range of 5 to 50 μmol/g. As used herein, the term alkyl refers to straight-chained such as propyl, branched or cyclic alkyls from 1 to 10 carbons and the term alkoxy refers to methoxy, ethoxy, propoxy or the like. Preferentially, beads are silanized with commercially available (mercaptopropyl)trimethoxysilane following published procedures (Heckel et al. 1998; Badley et al., 1989) to give (mercaptopropyl)Bead. The (mercaptopropyl)beads are alkylated with halogenomethyl(aryl)ₙ to give [(aryl)ₙmethylmercaptopropyl]beads. Examples of halogenomethyl(aryl)ₙ are taken from the groups of benzylchloride, 1-chloromethylnaphtalene, 2-bromomethylnaphtalene, chloromethylbiphenyl, diphenylmethylchloride, triphenylmethylchloride (trityl chloride) and the like. Preferentially, tritylchloride is reacted with (mercaptopropyl) beads in dichloromethane in the presence of triethylamine, affording (tritylmercaptopropyl) beads (T-Bead, FIG. 2). Remaining free silanol groups are end-capped by reaction with dialkyldialkoxysilane such as dimethyldimethoxysilane or trialkylsilylimidazole or a mixture of chlorotrialkylsilane and pyridine or the like. Preferably, trimethylsilylimidazole is used. The resulting end-capped T-beads can reversibly bind picomolar amount of primers when incubated with DMT-on primers or DMT-off primers in the presence of an appropriate binding buffer.

Preparation of non porous glass beads comprises the following steps:

(a) Increasing the bead specific surface using mechanical means, laser ablation or chemical means. Chemical means includes etching the glass surface with a solution of HF in water, followed by a treatment with an aqueous solution of sodium hydroxide. Preferably the said glass beads are sodalime beads,

(b) Silanizing the said beads with a silane bearing hydrophobic moieties L.

Preferably, the said silanes have formula such as (Y)₄₋ₙ(L)ₙsilanes wherein, (i) Y=alkoxy, halides, (ii) n is ranging from 1 to 3, n being a finite integer and, (iii) L=Alkyl-, Aryl- or (ArylX)alkyl- wherein Aryl are groups such as phenyl, benzyl, biphenyl, phenanthryl or trityl groups and X=N, NC(O), NC(S), O, or S, and (c) End-capping of remaining surface silanol groups using (trialkyl)imidazolesilane or dialkyldialkoxysilane.

### Bead Primer Binding:

Nucleic acids synthesized using 5′-dimethoxytrityl phosphoramidites are obtained either as their 5′-unprotected form (DMT-off primers) or as their 5′-DMT-on protected form (DMT-on primers), (U.S. Pat. No. 4,725,677). Upon cleavage from the solid supports and deprotection, primers are often desalted or purified. Desalting or purification operations are carried out using the nonpolar, hydrophobic beads described herein.

Upon cleavage from the solid support and primer deprotection, crude solutions of reverse and forward primers are mixed in roughly equimolar proportion. The resulting primer solution is either dried down or preferably diluted with an appropriate binding buffer. Diluting with a binding buffer eliminates the step of evaporating the primer solution before carrying out the primer binding step. Failures to dilute the primer solution with a binding buffer result in a weaker primer binding at the bead surface. In one embodiment, beads of the invention can be used to simultaneously desalt and normalize a solution of crude primers to yield primer bound beads. In another embodiment, beads of the invention can be used to simultaneously purify and normalize a solution of crude DMT-on primers to yield DMT-on bound beads. The process of normalizing describes the non covalent binding of 10 to 50 picomoles of each primer per bead and wherein each bead of the said plurality of beads binds roughly identical amount of primers. Identical binding between beads provides greater reproducibility between amplification reactions. Some factors involved in primer binding capacity are the ionic strength of the binding buffer, the contact time between said primer solution and beads, and the primer concentrations of the primer solutions.

In one embodiment, purification and normalized binding of DMT-on primers take place using T-beads. DMT-on primers reversibly bind through their 5′-dimethoxytrityl groups while truncated sequences, salts and trace organic impurities are not retained.

Truncated sequences refer to oligonucleotides which were not elongated during the primer synthesis and were subsequently capped. If a crude solution consisting of DMT-on forward and reverse primers is provided, the T-beads purify, bind and normalize the pair of primers or a plurality of pair of primers in a process comprising the following steps:

(a) Dilute the crude solution with an appropriate binding buffer. Binding buffers are made of aqueous solution containing high concentration of salts such as ammonium chloride, ammonium acetate, sodium chloride, sodium bromide sodium iodide and 0 to 10% of dimethylformamide or dimethylsulfoxide. For instance, binding buffer concentration is more than about 4 to 5 M Na ion, typically at pH ranging from 6.5 to 8.

Crude primer solutions are diluted in a 1:1 or 1:2 ratio respectively with the said binding buffer.

(b) Add a plurality of beads to the primer solution prepared in (a) and incubate the beads for a sufficient period of time until the normalized binding of each primer has been reached, and

(c) Detritylate the DMT-on bound primers with a protic acid to afford the corresponding DMT-off primer bound beads. The DMT-off primer bound beads are called primer bound beads. The DMT groups are cleaved using solutions of 1 to 5% dichloroacetic acid (DCA) or trichloroacetic acid (TCA) or trifluoroacetic acid (TFA) in water. Primer bound beads are washed, drained, and dried. Primer binding can take place in a few minutes to a few hours at room temperature. Time dependant conditions are not sequence-dependent for short sequences (e.g., 10 to 50 nucleotides) but function of the loading temperature, the primer concentrations and the binding buffer ionic strength. By way of example, the following procedure yields purified primer bound beads: (i) Prime T-beads with ethanol; (ii) Wash with 0.1 M triethylammonium acetate (TEAA), pH 7.0. (iii) Dilute the crude primer mixture with 30% NaCl in water and load the resulting mixture onto a plurality of primed beads; (iv) Wash with 0.1M TEAA, pH 7.0. (v) Detritylate the DMT-on primer bound beads with 2.5% dichloroacetic acid in water; (vi) Wash with 0.1M TEAA, pH 9.0.

In one embodiment, desalting and normalized binding of DMT-off primers using the bead of the invention take place in a process comprising the following steps: (i) diluting a crude DMT-off primer solution with a binding buffer, and (ii) adding a plurality of beads and leaving the said beads in contact with the solution prepared in (i) for a sufficient period of time until the optimal loading capacity of each bead has been reached. The resulting beads are then drained, washed and dried. One binding buffer composition useful to primer desalting and normalization is more than about 5 M Na ion at pH ranging from 5.5 to 8. After binding has occurred, the beads are washed thoroughly to remove salt, organic contaminants and unbound oligonucleotides.

Primer bound beads are dried and subsequently stored at temperature ranging from 4° C. to room temperature with no primer degradation being detected over a period of a few months. Primer bound beads of a same lot bind and deliver similar amount of primers thus ensuring a plurality of PCR reactions to be run overtime with reproducibility. This yields a convenient entry to diagnostic kits using identical primers over large numbers of PCR reactions. Primer bound beads eliminate the potential for pipeting errors and reduce contamination thus yielding lower repeat rates and less reagent wastage. Primer bound beads can be used as such to deliver primers in amplification reactions or can be further coated or silanized.

### Primer Delivery (Release) During Amplification Reactions

In an embodiment of the invention, the amplification reaction is a polymerase chain reaction (PCR) wherein at least one primer and, preferably both primers are delivered in situ by a primer bound bead. Other primer-based nucleic acid amplification methods can benefit from a bead carrying normalized primers included, but not limited to, the Ligase Chain Reaction (Barany, 1991). Primer bound beads are compatible with standard thermocyclers using 10 to 100 μL reaction volumes. A bead typically takes up a small volume, less than 2 μl of the total PCR reaction volume. For instance, a non porous sodalime bead of 1.5 mm diameter occupies a volume of less than 1.5 μL.

Primers are released from the beads when the temperature is first raised in the PCR cycle thus yielding a novel entry to hot start PCR by reducing room temperature mispriming. However, primers slowly leach from their primer bound bead when placed in an amplification buffer at room temperature for a long period of time. To further reduces non-specific amplifications, the present invention can be used with other methods of reducing non-specific amplification such as by using blocked primers bound to the beads. The particular method used is not a critical part of the present invention. In one embodiment, a bead carrying two normalized primers (reverse and forward) is transferred to a PCR tube containing DNA polymerase, target DNA and PCR buffer. The reaction mixture is subjected to multiple cycles of denaturation, annealing and elongation, resulting in the exponential amplification of the target DNA. In another embodiment, two beads carrying one primer each are transferred to a PCR tube containing DNA polymerase and PCR buffer. The reaction mixture is subjected to multiple cycles of denaturation, annealing and elongation, resulting in the exponential amplification of the target DNA. In yet another embodiment, a bead carrying a plurality of normalized primers (reverse and forward) is transferred to a PCR tube containing DNA polymerase and a plurality of DNA targets in a PCR buffer. The reaction mixture is subjected to multiple cycles of denaturation, annealing and elongation, resulting in the exponential amplification of the said plurality of DNA targets.

Amplification reactions carried out with primer bound beads can be monitored by measuring the increase in the total amount of double-stranded DNA in the reaction mixture and relies on the increased'fluorescence that ethidium bromide and other DNA binding labels exhibit when bound to double-stranded DNA. The measured fluorescence depends on the total amount of double-stranded DNA present and benefits from the lower non-specific amplification provided by primer bound beads.

Coating or silanization of primer bound beads Primer bound beads gradually release their bound primers in solution at room temperature and during the incubation step. Because the primers are incapable of being extended until they are released from the bead, further preventing primer leaching at room temperature insures higher specificity of the amplification reaction.

In one embodiment of the invention, primer bound beads are silanized by reacting the primers bound beads with solutions of silane containing hydrophobic moieties. Preferably, the said silanes are trialkylalkoxysilane or dialkylalkoxysilane or trialkylsilylimidazole dissolved in an organic solvent such as toluene, wherein alkyl groups include straight chained or branched hydrocarbons which are completely saturated. Preferentially, trimethylsilylimidazole (TMS) or dimethyldimethoxysilane (DMS) are used. The silanization step suppresses the room temperature leaching of primers into the amplification buffer. This in turn minimizes non-specific products and yield higher sensitivity amplification reactions. In another embodiment, primer bound beads are coated by incubating primers bound beads with solutions of polyalkylsiloxane containing hydrophobic moieties. Preferably, the said polyalkylsiloxane is polydimethylsiloxane dissolved in an organic solvent such as toluene.

In a preferred embodiment, the present invention provides a method for the amplification of a target nucleic acid using a coated or a silanized primer bound bead. PCR amplifications of fewer than 1000 copies of template are challenging and hampered by the synthesis of non-specific amplification products that consume the stock of primers and reduce the yield of amplicons. The use of a coated primer bound bead or a silanized primer bound bead minimizes non-specific amplification products, in particular primer-dimer, compared to amplifications carried out using primers that have been pipetted in the PCR buffer. The specificity of the polymerase chain reaction is notably compromised by primer-dimer formation early in the amplification process. A gradual and controlled release of primers provided by the coating or silanization step results in lower concentration of primers during the first amplification cycles and favor primer binding to the target nucleic acid and not to each other. This allows for a more efficient use of the DNA polymerase, deoxynucleoside triphosphates and other reaction components, to amplify the target nucleic acid and minimizes primer-dimer extension.

Coating or silanizing the primer bound bead further increase primer stability and nuclease resistance. Empirical selection of a coating with the desired stability from the class of compounds described can be carried out routinely by one of skill in the art following the guidance provided herein. The coated or silanized primer bound beads of the present invention are particularly useful in kinetic PCR because they not only reduce the amount of primer-dimer formed, but also delay the formation of detectable amounts of primer-dimer. Beads are useful for the development of PCR reactions, notably those in which the primers are not well designed and/or the reaction conditions are not optimal. A delayed primer-dimer formation until after a significant increase in target sequence has occurred enables independent monitoring of the amplification of target sequences and minimizes the interference from primer-dimers.

The present invention is to be described in further detail by referring to Examples, each of which is illustrative of an embodiment of the present invention and not limitative of the scope of the invention.

### EXAMPLES

The preparation of beads, the binding and purification of picomolar amounts of primer, the coating or silanization of primer bound beads and their use in amplification reactions are further described in the following examples. Many modifications and variations of bead preparations, bead coating and PCR use are possible in light of the above teaching. The following examples are not intended to be exhaustive or to limit the invention to the precise forms disclosed. They serve to illustrate the present invention and are not to be considered limitations thereof.

### Materials and Methods

Templates and primers were synthesized using 5′-DMT protected nucleotide cyanoethyl phosphoramidites on a Dr Oligo synthesizer (Biolytic Lab Performance).

After completion of a nucleic acid synthesis, the final 5′-DMT groups were left on or were deprotected yielding DMT-off primers. Primers were cleaved from their solid supports using butylamine: H₂O (1:3, 30 μL, 15 min) then deprotected using ammonium hydroxide (450 μL, 2 hrs, 75° C.). A sample (50 μL) is used to measure optical density and concentration of oligonucleotides are calculated in pmol/μl. Reverse and forward primers were analyzed by ion exchange HPLC (IEx-HPLC), quantified and mixed in equimolar proportion.

To illustrate the bead properties, TaqPol polymerase which is known to amplify non-specific hybridization taking place during the lower pre-amplification temperatures, was chosen. Amplification reactions were initiated with primers pipeted in solution or bound to a bead. The PCR products were analyzed without further processing by IEx-HPLC using the on-line UV detector (Hayward et al, 1996). The HPLC system comprised a 1090 HP, a column oven set at 55° C., a variable wavelength UV detector set at 260 nm. A Dionex column “BioLC DNAPac PA-100” was used for analysis. Injection volumes were 25 μL. The column was equilibrated in buffer A (0.1M TEAA, 20% ACN, pH 6.5) and eluted in a gradient of buffer B (2.5M NH₄Cl, 10% acetonitrile, pH 6.5). Following are the gradient tables used to analyze primers and PCR reactions.

HPLC gradient for primer analysis

| Time | % B | |
|---|---|---|
| (min) | buffer | ml/min |
| 0.00 | 0 | 0.50 |
| 0.50 | 5 | 1.00 |
| 4.00 | 30 | 1.00 |
| 4.50 | 70 | 1.00 |
| 5.00 | 70 | 1.00 |
| 5.50 | 0 | 1.00 |
| 6.50 | 0 | 1.00 |
| 7.00 | 0 | 0.50 |

HPLC gradient for PCR reaction analysis

| Time | % B | |
|---|---|---|
| (min) | buffer | ml/min |
| 0.00 | 0 | 0.50 |
| 0.50 | 5 | 1.00 |
| 5.00 | 25 | 1.00 |
| 8.00 | 35 | 1.50 |
| 8.50 | 50 | 1.00 |
| 9.00 | 50 | 1.00 |
| 9.50 | 0 | 1.00 |
| 10.00 | 0 | 0.50 |

### Example 1

### Preparation of Porous T-Beads

A thousand of porous glass beads (1 mm average size, 500 Å pore size) were put in dichloromethane (DCM, 2 mL). 3-Mercaptopropyltrimethoxysilane (10 mg) in DCM (1 mL) is added slowly to the beads. The beads are shaken gently for four days affording (mercaptopropylsilyl) beads. Triethylamine (25 nL) and tritylchloride (75 mg) in DCM (1 mL) are added and the beads are shaken gently at room temperature for two days. Trimethylsilylimidazole (50 μL) is added and the reaction mixture is shaken overnight. The T-Bead (Trityl-beads) are filtered, washed successively with acetone, methanol, water then acetone, and dried.

### Example 2

### Primer Binding to T-Beads

An Oligo Binding Solution (OBS) is prepared using the following solutions: (a) A

DMT-on primer solution, prepared by mixing the deprotected solution of forward primer with the deprotected solution of reverse primer in equimolar proportions. Dilution of the mix primer from their initial concentration to their working concentration is carried out using a solution of NH₄OH-DMF (15:5). The working primer concentration is 9 pmol of each primer/μl. (b) A binding buffer, prepared by dissolving 30% NaCl in water.

An OBS is prepared by mixing one volume of the DMT-on primer solution with one volume of the binding buffer. The final OBS concentrations were the following: 7.5% NH₄OH, 2.5% DMF, 15% NaCl and 4.5 pmol of each primer/μl.

Prior the binding step, T-beads are primed with ethanol (10 μl/bead), drained then washed with a solution of triethylammonium acetate [TEAA 0.1M, pH 7; 10 μl/bead)] and drained. Twenty primed T-beads were shaken with 150 μL of OBS for 30 min. The resulting DMT-on primer bound beads were washed twice with TEAA (0.1M at pH 7, 10 μL/bead) and drained. DMT removal was carried out with a solution of 2.5% of trichloroacetic acid in water (10 μl/bead). The beads were shaken for 60 sec then drained. The resulting primer bound beads were washed twice with TEAA (0.1M at pH 7, 10 μL/bead) then dried. Primer bound beads are dried before being used in a PCR reaction. They can be further silanized or coated as described in examples 3 and 4, respectively.

Primer standard curve calibrations were prepared by injecting and plotting known amounts of primer against the resulting primer HPLC peak integration area. Primers binding to a T-bead were quantified by IEx-HPLC after eluting the primers with a solution of 40% acetonitrile in water.

Example 3: Silanization of primer bound beads Primer bound beads are reacted with a solution of 5% trimethylsilylimidazole

(TMS) in toluene (10 μl/bead) for 24 hrs. After the reaction period, the solution of TMS in toluene is removed by filtration, and the beads were left at room temperature to dry.

### Example 4

### Coating of Primer Bound Beads

Primer bound beads are coated with a solution of 5% polydimethylsiloxane in toluene (10 μl/bead) for 20 min. After the reaction period, the solution of polydimethylsiloxane in toluene is removed by filtration, and the beads were left at room temperature to dry.

### Example 5

### Comparative Mispriming Studies

The role played by the beads in hot start amplifications was emphasized by using primers FW21 and RV22 containing a large number of G and C bases at the 3′-end. Those primers were designed to ensure mispriming even under suitably stringent amplification conditions through formation of six 3′-terminal GC pairing. Indeed, addition of both primers at 92° C. in a manual hot start experiment did not eliminate mispriming. The nucleotide sequences of the reverse and forward primers and the 107-mer template are as follow, oriented in the 3′ to 5′ direction: FW21 (SEQ ID NO: 1): CGGCGGGGTTCCGTGTCGAAC; RV22 (SEQ ID NO: 2): CCGCCGGAC GAGACATAGCACG; 107-mer Template (SEQ ID NO: 3): CAAGCTGTGCCTTGGGGCGGCGGCTTGGGGCATGGACATTGAGAATTTGGATCTG ACTTCTTTCCTTCTATTCGACCTCGACAGGCGGCCTGCTCTGTATCGTGC. Amplifications of the said 107-mer template were carried using either primers in solution or primer bound beads. Amplifications were carried out in 50 μl reactions\volumes containing the following reagents: Copies of a 107-mer template DNA ranging from 10⁷ to 10¹⁰ copies, 0.2 μM of each primer (10 pmoles), 200 μM each dATP, dCTP, and dGTP, 600 μM dTTP, MnOAc (25 mM, 5 μl), 1 unit of TaqPol DNA polymerase. In the case of reactions run with primer bound beads, two μl of water were added instead of the primers in solution to complete the 50 pt reaction volume.

The reaction cycle comprised preheating 5 min at 95° C. followed by 30 cycles comprising denaturation (94° C., 30 s), annealing (69° C., 30 s), extension (72° C., 30 s). The final cycle was followed by an additional 7 min at 72° C. to ensure complete extension.

Analyses of PCR products (Amplicons, Primer-dimers) and primers were conducted by Ion-Exchange HPLC. Results comparing PCR using primers in solution vs.

PCR using a primer bound bead are provided in Table 1, and are reported as the integration peaks of the PCR products. The data indicate that a primer bound bead lowers the formation of primer-dimers significantly. The beneficial effect of a primer bound bead vs. primers in solution was seen as well when a template-free reaction was carried out by delaying the formation of primer-dimers.

TABLE 1
The use of a primer bound Bead reduces non-specific amplification
products and increases Amplicons yields (as measured by integrating
the HPLC area of their peaks) compared to amplifications carried out
using primers that have been pipeted in solution.

| | Primer bound Bead | | Primers in Solution | |
|---|---|---|---|---|
| Copies Template | Primer-dimer | Amplicons | Primer-dimer | Amplicons |
| 10E7 | 60 | 36 | 122 | 12 |
| 10E8 | 22 | 61 | 112 | 23 |
| 10E9 | 16 | 103 | 89 | 67 |
| 10E10 | 8 | 191 | 64 | 171 |

### Example 6

### Hot Start Amplification Using Silanized Primer Bound Bead

The nucleotide sequences of reverse primer (SEQ ID NO: 4), forward primer (SEQ ID NO: 5) and 106-mer template (forward sequence: SEQ ID NO: 6) are as follow, oriented in the 3′ to 5′ direction: (SEQ ID NO: 4): CCCGATACAGAGCAGAGGCG; (SEQ ID NO: 5): CAAGCTGTGCCTTGGGTGGC; (SEQ ID NO: 6); CAAGCTGTGCCTTGGGTGGCTTTGGGGCATGGACATTGAGAATTTGGATCTG

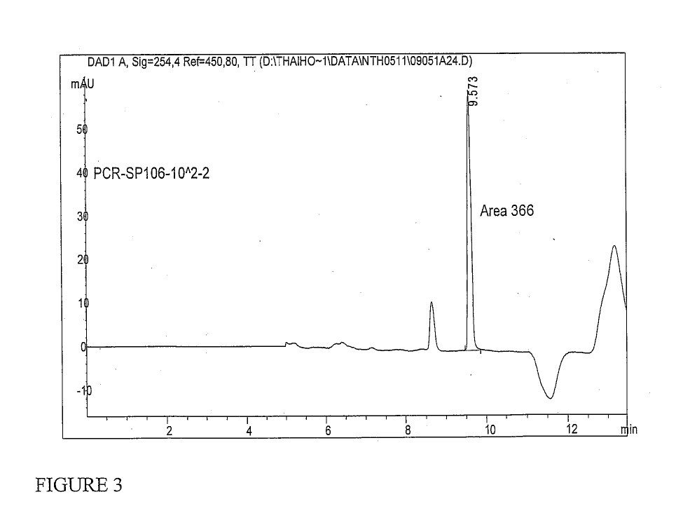

ACTTCTTTCCTTCTATTCGAGATCTCCTCGACACCGCCTCTGCTCTGTATCGG. The 3′-terminal sequences were designed with a run of four or less 3′-terminal G or C bases in order to minimize mispriming. Amplifications of 10 and 100 copies of the 106-mer template were carried out using either primers in solution or using a trimethylsilylimidazole silanized T-bead carrying reverse and forward primers, under the following PCR conditions: one initial cycle of denaturation at 94° C. for 3 min; 30 cycles of denaturation at 95° C. for 20 s, annealing at 62° C. for 30 s, and extension at 72° C. for 30 s. When primers were pipeted in solution, amplification of 100 copies of templates yielded primer-dimer (identified as their HPLC peak at 8.6 min) and amplicon (Peak at 9.5 min). No primers at 6.4 min remained (see FIG. 3). When a silanized primer bound

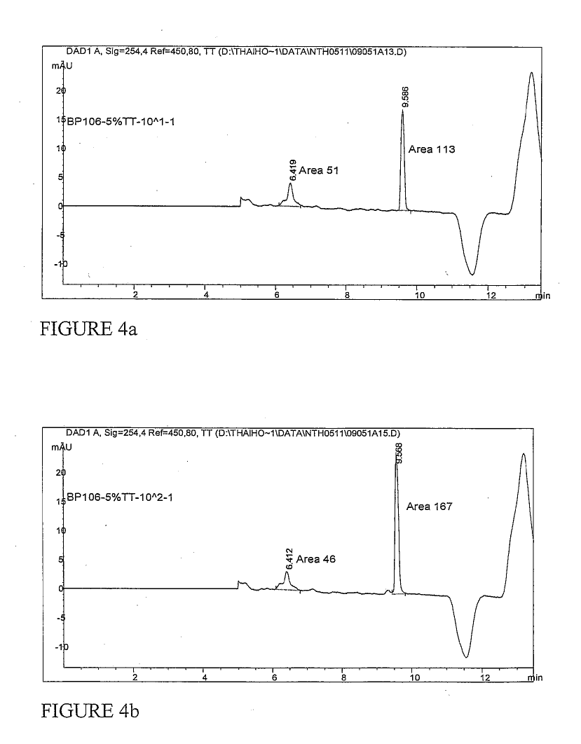

Bead was used, no primer-dimer peak was observed at 100 copies of template (See FIG. 4a). Likewise, amplification of 10 copies of template with a silanized primer bound bead of the same lot yielded amplicon (peak at 9.4 min); no primer-dimer peak was observed (See FIG. 4b). Remaining primers were observed as a peak eluting at 6.4 min.

### Example 7

Evidence of Gradual Release of Primers from Silanized Primers Bound Bead (Seq ID 4 and 5) primer bound beads were silanized with 5% trimethylsilylimidazole in toluene (TMS) or 5% dimethyldimethoxysilane in toluene (DMS), as described in Example 3 and 4, respectively. A (DMS) or (TMS) primer bound bead was placed in a PCR buffer (50 μL). After each step, the PCR buffer was analyzed by ion exchange HPLC, discarded and replaced with 50 μL of new PCR buffer. Step 1: At room temperature, for 15 min. Step 2: After the preheating step; 94° C., 5 min. Step 3:

After running three PCR cycles. Step 4: After running another three PCR cycles. Step 5: After running another five PCR cycles. Step 6: Elution of remaining bound primers by heating with 40% acetonitrile /1% t-butylamine in water at 94° C. for 15 min. Primer releases in the PCR buffers were quantified by integrating their Ion Exchange peak area after each step. Each step release is reported in the Table 2 as a % of the total primer release.

TABLE 2
Evidence of gradual release of primers over more than ten PCR
cycles for DMS or TMS silanized primer bound beads.

| Beads | Step 1 | Step 2 | Step 3 | Step 4 | Step 5 | Step 6 |
|---|---|---|---|---|---|---|
| TMS | 4% | 12% | 18% | 21% | 25% | 20% |
| DMS | 4% | 30% | 22% | 22% | 16% | 7% |

Hydrophobized glass beads facilitate purification and normalization of crude primer solutions. They simplify nucleic acid amplification techniques by requiring minimal reagent preparation and operator involvement. Primers are released from the beads when the temperature is first raised in the PCR cycle, thus yielding a new entry to hot start PCR by reducing room temperature mispriming. Gradual release of primers in solution is further controlled by coating or silanizing primer bound beads. Primer-dimer free amplifications indicate that the post-binding silanization step prevents the room temperature leaching of primers under the reaction conditions used and allows a gradual release of primers to take place over a large number of PCR cycles.

1. A method comprising:
a) providing a bead having a normalized, non-covalently bound oligonucleotides, and b) gradually releasing the oligoneucleotides from the bead during an enzymatic reaction.

2. The method of claim 1, including an initial step of preparing the bead by purifying a crude solution of oligonucleotides by capture of a normalized amount of purified oligonucleotides on said bead.

3. The method of claim 2, wherein purifying includes desalting a crude solution of oligonucleotides and capture of a normalized amount of oligonucleotides.

4. The method of claims 1, wherein said normalized, non-covalently bound oligonucleotides consists of a picomolar amount of a primer.

5. The method of claim 1, wherein the bead is made of nonporous glass having a nonpolar, hydrophobic surface.

6. The method of claim 1, wherein the bead is made of porous glass having nonpolar, hydrophobic surfaces.

7. The method of claim 1, wherein the bead is made of porous or nonporous glassing having non-polar hydrophobic surface including linear or branched alkyl chains selected from C₃ to C₂₀, aryl, benzyl, naphtyl, phenanthryl and trityl groups.

8. The method of claim 1, further including an initial step of preparing the bead having a normalized, non-covalently bound oligonucleotides by reacting glass beads with silanes, wherein said silanes include at least one selected from a group consisting of trialkyl(alkoxy)silane, dialkyl(dialkoxy)silane, aryl(alkyl)(dialkoxy)silane, aryl(trialkoxy)silane, diaryl(dialkoxy)silane, triaryl(alkoxy)silane, fluoroalkylalkoxysilane and alkylbis(trialkoxysilane).

9. The method of claim 1, wherein said bead includes a non-polar hydrophobic surface including surface (Aryl)-X-alkyl groups wherein said Aryl is selected from a group consisting of phenyl, benzyl, biphenyl, naphtyl, trityl, X═C, O, S, SC(O)N, OC(0)N, NC(0), and N; and n =1 to 3, n being a finite integer equal or greater than one.

10. The method of claim 2, wherein purifying a crude solution of oligonucleotides includes binding oligonucleotides having a 5′-hydrophobic moiety from the crude solution containing at least one contaminant.

11. The method of claim 10, wherein the 5′-hydrophobic moiety is a 4,4′-dimethoxytrityl group (DMT) and said contaminant is a DMT-off nucleic acid.

12. The method of claim 1, wherein the normalized, non-covalently bound oligonucleotides includes at least one reverse primer and a forward primer.

13. The method of claim 12, wherein the normalized, non-covalently bound oligonucleotides further includes at least one nucleic acid probe.

14. The method of claim 10, further including after capture of a normalized amount of purified oligonucleotides on said bead i) exposing said bead to a solution containing 10 to 100% of methanol, ethanol, acetonitrile or acetone in water; and
ii) subsequently exposing said bead to a solution containing 0.1M to 1.0 M concentration of monoalkylammonium, dialkylammonium, trialkylammonium acetate at pH ranging from 6 to 9.5.

15. The method of claims 10 further comprising diluting the crude solution with a binding buffer and mixing a resulting solution with a plurality of primed beads yielding DMT-on oligonucleotide bound bead.

16. The method of claim 15, further including washing the DMT-on oligonucleotide bound bead with a washing buffer that removes contaminants but not bound DMT-on oligonucleotides.

17. The method of claim 15, further comprising a step of cleaving DMT groups of the said bound DMT-on oligonucleotide yielding DMT-off oligonucleotide bound bead.

18. The method of claim 15, wherein the binding buffer is added in 2:1 to 1:2 volume per volume to the crude solution.

19. The method of claim 1, wherein said bead having a normalized, non-covalently bound oligonucleotides includes a bead having a non-covalently bound DMT-off oligonucleotide, and further including coating said bead with solution of polyalkylsiloxane to yield coated oligonucleotide bound beads.

20. The method of claim 1, wherein said bead having a normalized, non-covalently bound oligonucleotides includes a bead having a non-covalently bound DMT-off oligonucleotide, and further including treating the bead to yield silanized oligonucleotide bound beads.

21. The method of claims 1, wherein said beads comprise non porous glass beads having an average diameter of about 0.5 to 1.5 mm.

22. The method of claims 1 wherein said bead is a porous glass bead having pores ranging from 100 to 1000 Å and having an average diameter of 0.05 to 1.5 mm.

23. A method of hot start PCR comprising:
a) providing a bead having normalized, non-covalently bonded primers on a bead surface; and
b) gradually releasing the primers during temperature cycling wherein said bead is coated with a coating that inhibits subsequently applied higher temperature.

24. The method of claim 23, wherein said bead has a picomolar amount of primer.

25. The method of claim 23, in which said bead is glass having a nonpolar, hydrophobic surface.

26. A bead including:
a coating of non-covalently bound primers; and
a means for gradually releasing said primers from said bead during temperature cycling.


SEQUENCE LISTING (6 sequences)

SEQ ID NO: 1 - DNA, 21 bp, Artificial Sequence
  Forward primer for amplification of template named SEQ ID NO 3
caagctgtgccttggggcggc

SEQ ID NO: 2 - DNA, 22 bp, Artificial Sequence
  Reverse primer for amplification of template named SEQ ID NO 3
gcacgatacagagcaggccgcc

SEQ ID NO: 3 - DNA, 102 bp, Artificial Sequence
  Template
cgtgctatgtctcgtccggcggacagctccagcttatcttcctttcttcagtctaggtttaagagttacaggtacggggttcggcggggttccgtgtcgaac

SEQ ID NO: 4 - DNA, 20 bp, Artificial Sequence
  Reverse primer for amplification of template named SEQ ID NO 6
gcggagacgagacatagccc

SEQ ID NO: 5 - DNA, 20 bp, Artificial Sequence
  Forward primer for amplification of template named SEQ ID NO 6
cggtgggttccgtgtcgaac

SEQ ID NO: 6 - DNA, 105 bp, Artificial Sequence
  Template
ggctatgtctcgtctccgccacagctcctctagagcttatcttcctttcttcagtctaggtttaagagttacaggtacggggtttcggtgggttccgtgtcgaac